(19) United States
(12) Patent Application Publication
(10) Pub. No.: US 2007/0149779 A1
(43) Pub. Date: Jun. 28, 2007

(54) PRIMIDONE PROCESS AND COMPOSITIONS

(76) Inventors: Eswaraiah Sajja, Hyderabad 500 072, Andhra Pradesh (IN); Sudeep Kumar Agrawal, Raipur 492 010, Chhattisgarh (IN); Alagumurugan Alagarsamy, Tuticorin, Tamil Nadu (IN); Ravindar Reddy Koppera, Hyderabad 500 072, Andhra Pradesh (IN); Madhusudhan Reddy Ganta, Hyderabad 500 072, Andhra Pradesh (IN); Ramesh Babu Konda, Karim Nagar 505 472, Andhra Pradesh (IN); Ganesh Varanasi, Hyderabad 500 060, Andhra Pradesh (IN); Indu Bhushan, Hyderabad 500 072, Andhra Pradesh (IN); Mailatur Sivaraman Mohan, Hyderabad 500 072, Andhra Pradesh (IN); Asif Anwar, West Champaran, 845 438 Bihar (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/612,553

(22) Filed: Dec. 19, 2006

Related U.S. Application Data
(60) Provisional application No. 60/747,942, filed on May 23, 2006.
(60) Provisional application No. 60/747,950, filed on May 23, 2006.

(30) Foreign Application Priority Data
Jan. 10, 2006 (IN) 46/CHE/2006
Dec. 26, 2005 (IN) 1911/CHE/2005

(51) Int. Cl. C07D 239/62

(52) U.S. Cl. 544/299

(57) ABSTRACT

The present invention relates to a process for the preparation of primidone. It also relates to compositions comprising primidone of desired particle size distribution, processes for the preparation of such particles and processes for the preparation of the compositions.

### INTRODUCTION TO THE INVENTION

The present invention relates to a process for the preparation of primidone. It also relates to compositions of primidone or its pharmaceutically acceptable salts, solvates, polymorphic forms like crystalline or amorphous forms, or mixtures thereof, of desired particle size, processes for the preparation of such particles and processes for the preparation of compositions thereof.

Primidone is chemically known as 5-ethyldihydro-5-phenyl-4,6 (1H, 5H pyrimidinedione) and has the structural Formula I.

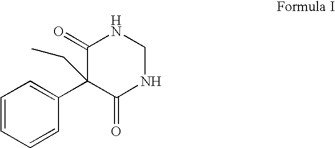

Primidone is used as anticonvulsant drug and is commercially available under the brand name MYSOLINE® as tablets containing 50 or 250 mg of primidone. It is very slightly soluble in water (0.6 mg/mL) and in most organic solvents.

Primidone tablets has an official monograph in United States Pharmacopeia 28, United States Pharmacopeial Convention, Inc., Rockville, Md., 2005 (“USP”). The dissolution requirement as per USP 28 is not less than 75% of the labeled amount of primidone should dissolve in 60 minutes in 900 ml of purified water, using Dissolution Test 711 apparatus 2 at 50 rpm.

Particle size and particle size distribution of the active ingredient plays an important role in selection of dosage form, processability of dosage form, dissolution and bioavailability. This role attains greater significance with increasing amounta of active ingredient in the unit dosage form. Parameters like flow, compressibility, hardness, friability and uniform content are critically affected by particle size. The tendency for a powder mixture to segregate can be reduced and flow properties can be improved by maintaining uniform particle size distribution in a specified range.

The rate of dissolution of poorly soluble drugs like primidone is a rate-limiting factor in its absorption by the body. A reduction in the particle size is expected to increase the dissolution rate of such compounds through an increase in the surface area of the solid phase that is in contact with the liquid medium, thereby resulting in an enhanced bioavailability of the compositions containing such compounds.

Particle size and particle size distribution are also important to the compression characteristics of a granulation (The Theory and Practice of Industrial Pharmacy, Ed. Lachman, L et al., Varghese Publishing House, Mumbai, 3^{rd} Edition, 692). Larger particle size results in segregation, improper granulation and lack of compressibility for certain active ingredients. Decreasing the particle size of active ingredient produces tablets of increased strength as well as a reduced tendency for lamination (Modern Pharmaceutics, Ed. Banker G. S and Rhodes C. T., Marcel Dekker Inc., 3^{rd} Edition, Volume 72, 335). But, an increase in strength causes disintegration and dissolution problems. Moreover, decrease in particle size beyond a certain limit causes problems like weight variation, poor content uniformity, stability and difficulty in handling (Pharmaceutical dosage forms: Tablets, Ed. Lieberman et al., Marcel Dekker Inc., 2^{nd} Edition, Volume 1, 5). Selection of a suitable particle size and a particle size distribution poses challenges to the formulators to design a formulation with all desired physico-chemical properties. It is generally not possible to predict the exact particle size and distribution that results in good physicochemical properties of active ingredient, good processability during formulation and a composition that meets pharmacopoeial dissolution and bioavailability criteria, as different drugs show different dissolution characteristics with a reduction in the particle size. The problem is further complicated by the fact that the same compound may exist in more than one crystalline form, each of which could further have a different dissolution profile.

U.S. Pat. No. 2,576,279 discloses a process for the preparation of primidone and claims primidone.

U.S. Pat. Nos. 2,676,176 and 3,165,459 disclose various processes for the preparation of primidone.

Great Britain Patent No. 666,027 discloses a process for the preparation of primidone.

It remains desirable to provide a simple, industrially feasible, inexpensive, and scaleable process for the preparation of primidone of Formula I.

Also primidone with higher particle size has shown poor compressibility index and resulted in processability issues such as low hardness, capping and lamination.

Thus, the present invention also addresses the need for compositions comprising primidone with desired particle size to meet required in vitro dissolution and in vivo absorption profiles.

### SUMMARY OF THE INVENTION

The present invention relates to a process for the preparation of primidone. It also relates to compositions of primidone of desired particle size, processes for the preparation of such particles and processes for the preparation of compositions thereof.

An aspect of the present invention provides for a process for the preparation of primidone, comprising the steps of:

i) reacting diethyl-2-ethyl-2-phenyl malonate compound of Formula IV with the thiourea compound of Formula III in the presence of sodium methoxide to afford the 5-phenyl-5-ethyl-2-thiobarbituric acid compound of Formula II; and

ii) desulfurizing the 5-phenyl-5-ethyl-2-thiobarbituric acid compound of Formula II with an excess of Raney nickel in presence of a suitable solvent to afford the primidone compound of Formula I.

An aspect of the present invention provides pharmaceutical compositions comprising primidone particles having a particle size D₉₀ less than about 50 μm.

Another aspect of the present invention provides for processes for preparation of primidone particles having a particle size D₉₀ less than about 50 μm, and pharmaceutical compositions thereof.

In an embodiment, the pharmaceutical compositions of the present invention include tablets comprising primidone particles having a particle size D₉₀ less than about 30 μm.

An aspect of the invention includes a process for preparing primidone, comprising reacting a compound having a formula: 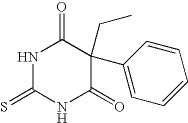
with Raney nickel, in an amount about 7 to about 20 times a weight of the compound, in a solvent.

Another aspect of the invention includes a process for preparing primidone, comprising reacting a compound having a formula: 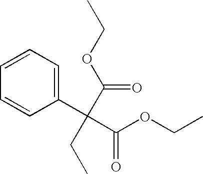
with thiourea in the presence of sodium methoxide, to form an intermediate compound having a formula: 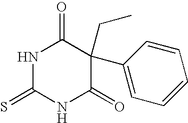
and further reacting an intermediate compound with Raney nickel, in an amount about 7 to about 20 times a weight of the intermediate compound, in a solvent to form primidone.

### DETAILED DESCRIPTION OF THE INVENTION

The present invention relates to a process for the preparation of primidone. It also relates to compositions of primidone of desired particle size, processes for the preparation of such particles and processes for the preparation of compositions thereof.

An aspect of the present invention provides a process for the preparation of primidone, comprising the steps of:

i) reacting diethyl-2-ethyl-2-phenyl malonate compound of Formula IV with the thiourea compound of Formula III 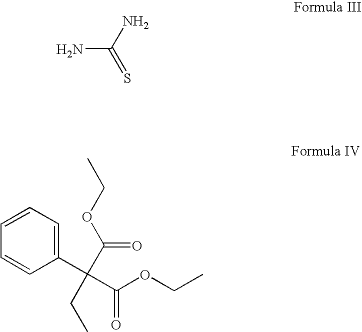
in the presence of sodium methoxide to afford the 5-phenyl-5- ethyl-2-thiobarbituric acid compound of Formula II; and

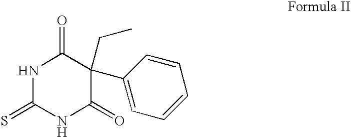

ii) desulfurizing the 5-phenyl-5-ethyl-2-thiobarbituric acid compound of Formula II with an excess of Raney nickel in the presence of a suitable solvent to afford primidone of Formula I.

Step i) involves reaction of the diethyl-2-ethyl-2-phenyl malonate of Formula IV with thiourea of Formula III to afford 5-phenyl-5-ethyl-2-thiobarbituric acid of Formula II, in the presence of sodium methoxide and a suitable solvent.

Suitable solvents that can be used for the preparation of the compound of Formula II include but are not limited to: alcohols such as methanol, ethanol, isopropanol, and the like; nitrile solvents such as acetonitrile, propionitrile and the like; aprotic polar solvents such as N,N-dimethylformamide (DMF), dimethylsulfoxide (DMSO), N,N-dimethylacetamide (DMA) and the like; ethers such as dimethyl ether, diethyl ether, di-isopropyl ether, methyl tertiary butyl ether, tetrahydrofuran, 1,4-dioxane, and the like; hydrocarbons such as toluene, xylene, n-hexane, n-heptane, cyclohexane and the like; or mixtures thereof without limitation.

Suitable temperatures that can be employed for the preparation of the compound of Formula II can range from about 10° C. to about 100° C., or from about 20° C. to about 80° C., or the reflux temperature of the solvent used.

In an embodiment, slow heating to reflux, such as over a period of 2-10 hours, during the preparation of the compound of Formula II results in high yield and purity of the primidone.

After completion of the reaction, the reaction suspension pH is adjusted to about 6 to 9, or from about 7 to 8, using an acid to precipitate the product.

Suitable acids that can be used include but are not limited to hydrochloric acid, hydrobromic acid, sulphuric acid and the like, mixtures thereof and their combinations in various proportions with water.

Optionally, the compound of Formula II is isolated by removing the solvent from the reaction mixture.

Solvent can be removed using conventional techniques like distillation, evaporation and the like.

In one embodiment of the present invention, the compound of Formula II is isolated by subjecting the reaction mass to distillation to remove the solvent.

Suitable techniques, which can be used for the distillation, include heating the reaction mixture to reflux or by distillation using a rotational evaporator device such as a Buchi Rotavapor, and the like.

Optionally the obtained solid material is further dried using any technique such as fluid bed drying (FBD), spin flash drying, aerial drying, oven drying or other techniques known in the art at temperatures of about 40° C. to 80° C., or from about 60° C. to 75° C., with or without application of vacuum and/or under inert conditions.

Step ii) involves desulfurizing the 5-phenyl-5-ethyl-2-thiobarbituric acid compound of Formula II with an excess of Raney nickel in a suitable solvent to afford primidone of Formula I

Suitable solvents that can be used for the preparation of the compound of Formula I include but are not limited to: ethers such as dimethyl ether, diethyl ether, diisopropyl ether, methyl tertiary-butyl ether, tetrahydrofuran, 1,4-dioxane and the like; hydrocarbons solvents such as toluene, xylene, n-hexane, n-heptane, cyclohexane and the like; aprotic polar solvents such as N,N-dimethylformamide (DMF), dimethylsulfoxide (DMSO), N,N-dimethylacetamide (DMA) and the like; aromatic hydrocarbons such as toluene, n-hexane, and the like; and mixtures thereof in various proportions without limitation.

It has been surprisingly found that using an excess of Raney nickel leads to high yields. Suitably a weight ratio of Raney nickel to the compound of Formula II between about 7 to about 20, or about 9 to about 11, is used.

Suitable temperatures that can be employed for the preparation of the compound of Formula I in step b) can range from about −5° C. to about 80° C., or from about 0° C. to 70° C.

After the completion of the reaction the reaction mass is filtered and filtrate can be concentrated under reduced pressure, such as to a volume of 10 to about 12% of the initial volume of the filtrate. The concentrate thus obtained is subjected to cooling to isolate the compound of Formula I a solid.

The solid material containing the compound of Formula I is isolated using any technique known in the art for solid-liquid separation and examples include decantation, vacuum filtration, gravity filtration, centrifugation, and removing the solvent either by subjecting to distillation or by evaporating the solvent.

In an embodiment of the present invention, the solid is separated by filtering the reaction mass either by applying vacuum or without applying vacuum.

In an embodiment, the compound of Formula I that is obtained according to the present invention can be optionally purified to wash out any residual impurities.

Purification can be carried out by providing a slurry of primidone in a suitable solvent at a suitable temperature, followed by isolating the pure compound of Formula I.

When the slurry is prepared by suspending primidone in a suitable solvent, any form of primidone such as any crystalline or amorphous form, including any salts, solvates and hydrates may be utilized for preparing the slurry.

Suitable solvents include but are not limited to: alcohols such as methanol, ethanol, isopropyl alcohol, n-butanol and the like; ketonic solvents such as acetone, ethyl methyl ketone, methyl isobutyl ketone and the like; esters such as ethyl acetate, n-propyl acetate, isopropyl acetate, n-butyl acetate, t-butyl acetate and the like; nitrile solvents such as acetonitrile, propionitrile and the like; halogenated solvents such as dichloromethane, ethylene dichloride, chloroform and the like; ethers such as dimethylether, diethyl ether, diisopropyl ether, methyl tertiary-butyl ether tetrahydrofuran, 1,4-dioxane and the like; hydrocarbon solvents such as toluene, xylene, n-hexane, n-heptane, cyclohexane and the like; and mixtures thereof or their combinations with water in various proportions without limitation.

Suitable temperatures that can be employed for purification can range from about 0° C. to about 70° C.

The primidone solid can be recovered using conventional solid-liquid separation techniques, such as decantation, centrifugation, gravity filtration, pressure filtration, vacuum filtration, and the like.

The isolated solid product can optionally be dried using any technique, such as fluid bed drying, aerial drying, oven drying or other techniques known in the art at temperatures of about 25 to 100° C., or 50 to 60° C., with or without application of vacuum and/or under inert conditions.

Primidone prepared according to the process of the present invention contains about less than or equal to about 0.15% by weight of each the following impurities listed in European Pharmacopoeia 5^{th} edition, Volume 2, pages 2309-2310:

impurity A (Ethyl phenyl malonamide);

impurity B (Phenobarbital);

impurity C ((2RS)-2-phenylbutanamide);

impurity E ((2RS)-2-phenylbutanoic acid);

compound of Formula II;

less than or equal to about 0.1% by weight of any single unknown impurity; and

less than or equal to about 0.5% by weight of total impurities as measured by high performance liquid chromatography (“HPLC”).

Primidone obtained by the process of the present invention contains less than about 5000 ppm, or less than about 3000 ppm, or less than about 1000 ppm, of total residual organic solvents and less than about 200 ppm, or less than about 100 ppm, or less than about 50 ppm, of individual residual organic solvents as determined by gas chromatography.

The present invention further relates to compositions of primidone of a desired particle size, processes for the preparation of primidone of a desired particle size and processes for the preparation of compositions thereof.

A particle size distribution of D₅₀ as used herein is defined as the distribution where 50 volume percent of the particles are smaller than that size given. A particle size distribution of D₁₀ as used herein is defined as the distribution where 10 volume percent of the particles are smaller than that size given. A particle size distribution of D₉₀ as used herein is defined as the distribution where 90 volume percent of the particles are smaller than that size given. The D₅₀ value is considered to be a “mean particle size”. These values can be determined using readily available equipment, such as laser diffraction apparatus sold by Malvern Instruments Ltd., of Malvern, Worcestershire, United Kingdom.

In one embodiment of the invention, the pharmaceutical composition comprises a plurality of primidone particles having a particle size D₉₀ about 50 μm, or about 30 μm, or about 15 μm, or about 5 μm.

In another embodiment, primidone particles having a particle size D₉₀ of about 30 pm have been found to be particularly useful in the context of instant invention.

“Carr index” as used herein is defined as the percent compressibility, that is a percentage ratio of the difference between tapped bulk density and initial bulk density to tapped bulk density. Carr index values between 5 and 15% represent materials with excellent flowability, values between 18 and 21% represent fair flowability and values above 40% represent very poor flowability.

In an embodiment of the invention, the pharmaceutical composition comprises a plurality of primidone particles with defined particle size, and having a Carr index more than about 15%, or more than about 25%, or more than about 35%.

In another embodiment, primidone particles with defined particle size, and having a Carr index more than about 20% have been found to be particularly useful in the context of the instant invention.

It will be appreciated by those skilled in the art of particle size reduction that there are numerous known methods which can be applied to the production of primidone of defined particle size, such as fluid energy milling or micronizing, ball milling, colloid milling, roller milling, hammer milling, and the like.

In one embodiment, a micronizer or a fluid energy mill is used for size reduction for its ability to produce particles of small sizes in a narrow size distribution. Micronizers use the kinetic energy of collisions between particles suspended in a rapidly moving fluid (typically air or an inert gas) stream to cleave the particles. A typical process for the preparation of primidone of desired particle size using a micronizer comprises the following steps:
a) charging primidone into a micronizer under an inert atmosphere;
b) running the micronizer containing primidone of step a) for a desired period; and
c) recovering the primidone of desired particle size.

During micronization, an inert atmosphere may be created by use of inert gases such as nitrogen, argon, neon and the like.

The temperature during the micronization process can range from about 20-45° C.

The particles of primidone used for micronization can be single crystals, aggregates, agglomerates, amorphous and any combinations thereof.

The particle size distribution of primidone of the present invention can be determined by techniques such as, for example, light scattering, laser diffraction, Coulter counter measurement, or microscopy. Other techniques for the measurement of particle size are also acceptable.

As used herein, “composition” means a solid dosage form for administration to mammal that includes primidone of a defined particle size distribution. A solid dosage form is considered to “comprise” primidone having a particular particle size distribution when it has been prepared using such primidone, regardless of the ultimate particle sizes that might result from formulation processing operations such as granulation and tablet compression to prepare the final dosage form.

The compositions of the present invention can be formulated as solid oral dosage forms such as but not limited to tablets, capsules, suspensions, powders for suspensions, and the like. The tablet dosage form is found to be particularly suitable in the context of instant invention.

In an embodiment, primidone particles having defined particle size, with one or more pharmaceutically acceptable excipients are converted to granules using dry granulation or wet granulation or any other granulation techniques known in the art.

In an embodiment, the average hardness of the tablet compositions of present invention varies from about 5 to 30 kiloponds (“Kp”), or about 5 to 20 kiloponds. The hardness may be measured by any conventional hardness tester such as for example a Strong Cobb, Monsanto, VanKel (Varian), Erweka, Pfizer, Schleuniger, or Pharma hardness tester.

The compositions of the present invention may comprise pharmaceutically acceptable excipients such as, but not limited to, diluents, binders, disintegrants, colorants, anti-oxidants, sweeteners and film-forming agents. A person skilled in the art of development and manufacture of pharmaceutical solid oral dosage forms is aware of the factors involved in making a choice of different excipients. A given pharmaceutically acceptable excipient may have more than one of characteristics or properties and classification of excipients according to function is therefore somewhat arbitrary.

In an embodiment of the present invention, the pharmaceutical composition comprises primidone particles and disintegrants, which are co-granulated, and further compressed into tablets using pharmaceutically acceptable excipients. Non-limiting examples of suitable disintegrants include carboxymethyl cellulose calcium, croscarmellose sodium (e.g. Ac-Di-Sol®, PRIMELLOSE®), crospovidone (e.g. KOLLIDON®, POLYPLASDONE®), polacrilin potassium, pregelatinized starch, sodium starch glycolate (e.g. EXPLOTAB®), and the like. In particular, croscarmellose sodium, crospovidone and sodium starch glycolate have been found to be useful.

In a further aspect of the present invention, the pharmaceutical composition comprises primidone in the range of about 25 mg to about 400 mg, or about 50 mg to about 250 mg, per unit.

In context of the present invention, during the preparation of the pharmaceutical compositions into finished dosage form, one or more pharmaceutically acceptable excipients may optionally be used which include but are not limited to: diluents such as microcrystalline cellulose (MCC), silicified MCC (e.g. Prosolv™), microfine cellulose, lactose, starch, pregelatinized starch, mannitol, sorbitol, dextrates, dextrin, maltodextrin, dextrose, calcium carbonate, calcium sulfate, dibasic calcium phosphate dihydrate, tribasic calcium phosphate, magnesium carbonate, magnesium oxide and the like; binders or adherents such as acacia, guar gum, alginic acid, dextrin, maltodextrin, methyl cellulose, ethyl cellulose, hydroxyethyl cellulose, hydroxypropyl cellulose (e.g. KLUCEL®), hydroxypropylmethyl cellulose (e.g. METHOCEL®), carboxymethyl cellulose sodium, povidone (various grades of KOLLIDON®, PLASDONE®), starch and the like; plasticizers such as acetyltributyl citrate, phosphate esters, phthalate esters, amides, mineral oils, fatty acids and esters, glycerin, triacetin or sugars, fatty alcohols, polyethylene glycol, ethers of polyethylene glycol, fatty alcohols such as cetostearyl alcohol, cetyl alcohol, stearyl alcohol, oleyl alcohol, myristyl alcohol and the like; solvents that may be used in granulation or layering or coating include water methanol, ethanol, isopropyl alcohol, acetone, methylene chloride, dichloromethane and the like or mixtures thereof.

Pharmaceutical compositions of the present invention may further include any one or more of pharmaceutically acceptable glidants like talc; lubricants like sodium stearyl fumarate and magnesium stearate; opacifiers; colorants and other commonly used excipients.

The following examples illustrate certain specific aspects and embodiments of the invention and demonstrate the practice and advantages thereof. It is to be understood that the examples are given by way of illustration only and are not intended to limit the scope of the invention in any manner.

### EXAMPLE 1

### Preparation of 5-phenyl-5-ethyl-2-thiobarbituric acid (Formula II) in Acetonitrile

400 ml of acetonitrile and 41 g of sodium methoxide were charged in a clean and dry round bottom flask under nitrogen at about 28° C. and stirred for 10 minutes. 28.8 g of the thiourea compound of Formula III was added to the above obtained reaction solution and stirred for 10 minutes. 100 g of the diethyl 2-ethyl-2-phenylmalonate compound of Formula IV, was added and the resultant reaction mass was heated slowly to 76° C. over a period of 2 hours with simultaneous stirring. The thus obtained reaction mass was cooled 18° C. and the pH was adjusted to about 7.2 using 60 ml of 2 N aqueous hydrochloric acid solution. The solvent from the reaction mass was distilled completely at about 44° C. under vacuum. The resultant residue was cooled to about 30 ° C. followed by addition of 500 ml of water and 500 ml of n-hexane. The reaction suspension was stirred for about 60 minutes at 30° C. and the solid was separated by filtration. The solid obtained was washed with 200 ml of water and 200 ml of n-hexane. The solid obtained was dried at about 72° C. under vacuum for about 12 hours to afford 67 g (yield 71.35%) of the title compound.

### EXAMPLE 2

### Preparation of 5-phenyl-5-ethyl-2-thiobarbituric acid (Formula II) in Methanol

150 ml of methanol and 39 g of sodium methoxide were charged in a clean and dry round bottom flask under nitrogen at about 28° C. and stirred for 10 minutes. 28.8 g of the thiourea compound of Formula III was added and heated to 52° C. with simultaneous stirring for 15 minutes. 50 g of the diethyl 2-ethyl-2-phenylmalonate compound of Formula IV, was added and the resultant reaction mass was heated slowly to about 55° C. over a period of 2 hours with simultaneous stirring. The thus obtained reaction mass was cooled 23° C. and the pH was adjusted to about 5.4 using 60 ml of 2 N aqueous hydrochloric acid solution. The reaction suspension was stirred for about 60 minutes at 28° C. and the solid was separated by filtration. The solid obtained was washed with 300 ml of water and suction dried for 50 minutes. The solid obtained was dried at about 72° C. under vacuum for about 12 hours to afford 38 g (yield 81%) of the title compound.

### EXAMPLE 3

### Preparation of Primidone (Formula I)

1000 ml of tetrahydrofuran and 50 g of 5-ethyl-5-phenyl-2-thiobarbituric acid of Formula II were charged in a clean and dry round bottom flask followed by heating to about 45° C. 5 g of activated charcoal was charged followed by stirring for about 30 minutes. The reaction suspension filtered through celite and the celite bed was washed with 250 ml of tetrahydrofuran.

500 ml of tetrahydrofuran and 500 g of Raney nickel were added to a clean and dry round bottom flask at about 26° C. 50 g of 5-ethyl 5-phenyl-2-thiobarbituric acid dissolved in 250 ml of tetrahydrofuran was added to the above and the resultant reaction suspension was heated to reflux at 64° C. over about 5 hours with simultaneous stirring. The reaction suspension was filtered through a celite bed, followed by washing the celite with 2×250 ml of tetrahydrofuran. The combined filtrate was concentrated under reduced pressure to a volume of 10% of the original volume of the filtrate, followed by cooling to about 5° C. and stirring for about 90 minutes. Separated solid was filtered and washed with 50 ml of tetrahydrofuran. The above-obtained crude compound of Formula I was slurried in 160 ml of methanol at about 64° C. for about 60 minutes. The resultant suspension was cooled to 30° C. and stirred for about 60 minutes followed by filtering the separated solid. The solid obtained was washed with 32 ml of methanol and dried at about 67° C. under vacuum to afford 27.8 g of a pure form of the title compound with purity by HPLC 99.7%.

### EXAMPLE 4

### Process for Preparation of Primidone Having Desired Particle Size

2.8 kg of primidone was charged in a clean and dry micronizer under nitrogen atmosphere followed by running the micronizer at about 25-35° C. for about 1.5 hours to about 2 hours under nitrogen.

Micronized product (yield 2.69 kg) of was collected and analyzed for particle size using a Malvern particle size analyzer. The particle size distribution was: D₁₀=0.95 μm, D₅₀=2.73 μm, and D₉₀=5.72 μm.

### EXAMPLE 5

### Primidone tablets 50/250 mg Having Primidone Particle Size D₉₀ Less than 10 μm.

Quantity/Batch (g)

| Ingredients | 50 mg strength | 250 mg strength |
|---|---|---|
| Primidone* | 50 | 250 |
| Lactose monohydrate | 43.8 | 218.9 |
| Sodium starch glycolate | 2 | 10 |
| Methyl cellulose | 1 | 5 |
| Sodium lauryl sulphate | 0.25 | 0.7 |
| Dimethicone | — | 0.5 |
| Water | 20 | 13 |
| Sodium starch glycolate | 2 | 10 |
| Talc | 0.5 | 2.5 |
| Magnesium stearate | 0.5 | 2.5 |

*Particle size D₉₀ = 7.1 μm

Manufacturing Process:
1. Primidone, lactose and sodium starch glycolate were sifted through an ASTM 40 mesh sieve.
2. Methyl cellulose was dispersed in water.
3. Sodium lauryl sulphate was dissolved in the dispersion of step 2 with stirring.
4. The ingredients of step 1 were granulated using the solution of step 3.
5. The wet granules were dried in a fluidized bed dryer at 60° C. until the loss on drying was below 1.5% w/w.
6. The granules of step 5 were mixed with sodium starch glycolate and lubricated with talc and magnesium stearate in a blender for 5 minutes.
   The granules of step 6 were compressed into tablets using 6.35 mm round flat punches. The average hardness of the tablets of the composition was 8 Kiloponds.

### EXAMPLES 6-7

### Primidone Tablets 50 mg Having Different Drug Particle Sizes

Quantity/Batch (g)

| | | Example 7* |
|---|---|---|
| Ingredients | Example 6* | (Comparative) |
| Primidone | 50 | 50 |
| Lactose monohydrate | 10 | 10 |
| Microcrystalline cellulose | 5.7 | 5.7 |
| Methyl cellulose | 1.4 | 1.4 |
| Sodium lauryl sulphate | 0.7 | 0.7 |
| Water | 20 | 13 |
| Sodium starch glycolate | 1.4 | 1.4 |
| Talc | 0.4 | 0.4 |
| Magnesium stearate | 0.4 | 0.4 |

*Primidone particle size D₉₀ = 7.1 μm
**Primidone particle size D₉₀ = 307 μm

The manufacturing process was similar to that given in Example 5, except that during the granulation process sodium starch glycolate was replaced with microcrystalline cellulose.

Properties of the starting primidone and the primidone granules are given in the following table.

| | Primidone | | | |
|---|---|---|---|---|
| | Particle size | Particle size | Primidone Granules | |
| Parameter | D₉₀ 7.1 μm | D₉₀ 307 μm | Example 6 | Example 7 |
| Bulk Density, | 0.221 | 0.850 | 0.65 | 0.72 |
| (D_{b}) g/ml | | | | |
| Tapped density | 0.307 | 0.905 | 0.79 | 0.75 |
| (Dₜ) g/ml | | | | |
| Carr Index | 28% | 6.1% | 17.7% | 4% |

The data show that compositions made with lower particle size primidone result in granules with good compressibility, compared to granules formed from higher particle size primidone.

The average hardness of the tablets prepared in Example 6 was 7 Kiloponds.

Severe capping was observed during compression of tablets in Example 7.

### EXAMPLES 8-9

### Primidone Tablets 250 mg Having Different Drug Particle Sizes

mg/Tablet

| | | Example 9** |
|---|---|---|
| Ingredients | Example 8* | (Comparative) |
| Primidone | 250 | 250 |
| Lactose monohydrate | 50 | 50 |
| Microcrystalline cellulose | 28.5 | 28.5 |
| Methyl cellulose | 7 | 7 |
| Sodium lauryl sulphate | 3.5 | 3.5 |
| Water | 100 | 100 |
| Sodium starch glycolate | 7 | 7 |
| Talc | 2 | 2 |
| Magnesium stearate | 2 | 2 |

*Primidone particle size D₉₀ = 25.3 μm
**Primidone particle size D₉₀ = 307 μm

The manufacturing process was similar to that given in Example 5, except that during the granulation process sodium starch glycolate was replaced with microcrystalline cellulose.

The average hardness of the tablets prepared in Example 8 was 13 Kiloponds, and no capping was observed.

Severe capping was observed during compression of tablets in Example 9.

### EXAMPLE 10

### Primidone Tablets 50/250 mg Having Primidone Particle Size D₉₀ Less than 10 μm.

| | Quantity/Batch (Kg) | |
|---|---|---|
| | 50 mg strength | 250 mg strength |
| Ingredients | (250,000 tablets) | (150,000 tablets) |

GRANULATION

| | | |
|---|---|---|
| Primidone | 12.5 | 37.5 |
| Lactose monohydrate | 10.8 | 32.38 |
| Sodium starch glycolate | 0.75 | 2.25 |
| Ferric oxide USP | — | 0.06 |
| Methyl cellulose | 0.25 | 0.75 |
| Sodium lauryl sulphate | 0.06 | 0.19 |
| Water | 8 | 24 |

BLENDING AND LUBRICATION

| | | |
|---|---|---|
| Sodium starch glycolate | 0.25 | 0.75 |
| Talc | 0.19 | 0.56 |
| Magnesium stearate | 0.19 | 0.56 |

Manufacturing Process:
1. Primidone and lactose were sifted through an ASTM 30 mesh sieve; and sodium starch glycolate and ferric oxide (if used) were sifted through an ASTM 60 mesh sieve; and then were blended together.
2. Methyl cellulose was dispersed in water and sodium lauryl sulphate was dissolved in this dispersion with stirring.
3. The ingredients of step 1 were granulated using the binder solution of step 2 in a rapid mixer granulator.
4. The granules of step 3 were dried in a fluidized bed dryer at 60° C. until the loss on drying was below 1.5% w/w.
5. The dried granules were passed through an ASTM 20 mesh sieve.
6. The granules of step 5 were blended with sodium starch glycolate and then lubricated with talc and magnesium stearate (previously passed through an ASTM 60 mesh sieve) in a double cone blender for 10 minutes.
7. The granules of step 6 were compressed into tablets using following tooling:
   50 mg strength: 6.35 mm round flat punches,
      Average tablet weight 100 mg.
   250 mg strength: 11.5 mm round flat punches.
      Average tablet weight 500 mg.

Tablet Evaluation:

| Parameter | 50 mg Strength | 250 mg Strength |
|---|---|---|
| Hardness (Kp) | 5-7 | 9-14 |
| Disintegration time (minutes) | 3 | 7 |
| Friability (% w/w) | 0.2-0.3 | 0.4 |

In vitro release profile of the product of Example 10 in comparison with a commercial product was determined with the following parameters:
Media: Purified water
Volume: 900 ml.
Apparatus: USP apparatus type II (Paddle) from Test 711 Dissolution in United States Pharmacopeia 29, United States Pharmacopeial Convention, Inc., Rockville, Md. (2005).

Speed: 50 rpm

| | % Drug Released | | | |
|---|---|---|---|---|
| | | | | MYSO- |
| | Primidone | MYSOLINE ® | Primidone | LINE ® |
| Time | Tablets 50 mg | Tablets | Tablets 250 mg | Tablets |
| (minutes) | (Example 10) | 50 mg | (Example 10) | 250 mg |
| 0 | 0 | 0 | 0 | 0 |
| 15 | 89 | 95 | 68 | 57 |
| 30 | 95 | 98 | 90 | 93 |

### EXAMPLE 11

### Primidone Tablets 50 mg and 250 mg.

| | |
|---|---|
| Ingredients | Composition (% w/w) |

GRANULATION

| | |
|---|---|
| Primidone | 10-15 |
| Lactose monohydrate | 10-15 |
| Sodium starch glycolate | 0.5-1 |
| Ferric oxide USP | 0-0.02 |
| Methyl cellulose | 0-0.5 |
| Sodium lauryl sulphate | 0-0.02 |

BLENDING AND LUBRICATION

| | |
|---|---|
| Sodium starch glycolate | 0-0.5 |
| Talc | 0.1-0.25 |
| Magnesium stearate | 0.1-0.25 |

Manufacturing process is similar to that described in Example 10.

1. A process for preparing primidone, comprising reacting a compound having a formula: 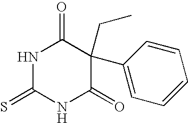
with Raney nickel, in an amount about 7 to about 20 times a weight of the compound, in a solvent.

2. The process of claim 1, wherein an amount of Raney nickel is about 9 to about 11 times a weight of the compound.

3. The process of claim 1, wherein reacting occurs in a solvent comprising tetrahydrofuran.

4. The process of claim 1, wherein reacting occurs at a reflux temperature of a solvent.

5. The process of claim 1, wherein a compound having a formula: 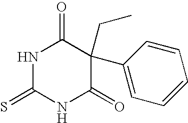
is prepared by reacting a compound having a formula: 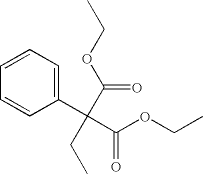
with thiourea in the presence of sodium methoxide.

6. The process of claim 5, wherein reacting with thiourea occurs in a solvent comprising acetonitrile or methanol.

7. A process for preparing primidone, comprising reacting a compound having a formula: 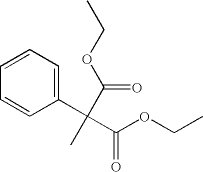
with thiourea in the presence of sodium methoxide, to form an intermediate compound having a formula: 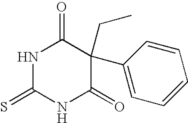
and further reacting an intermediate compound with Raney nickel, in an amount about 7 to about 20 times a weight of the intermediate compound, in a solvent to form primidone.

8. The process of claim 7, wherein reacting with thiourea occurs in a solvent comprising acetonitrile or methanol.

9. The process of claim 7, wherein an amount of Raney nickel is about 9 to about 11 times a weight of an intermediate compound.

10. The process of claim 7, wherein reacting with Raney nickel occurs in a solvent comprising tetrahydrofuran.

11. The process of claim 7, wherein reacting with Raney nickel occurs at a reflux temperature of a solvent.

12. A pharmaceutical composition comprising primidone particles having a particle size D₉₀ no greater than about 30 μm.

13. The pharmaceutical composition of claim 12, wherein primidone particles have a Carr index at least about 20 percent.

14. The pharmaceutical composition of claim 12, comprising tablets having a hardness about 5 to 30 kiloponds.

15. The pharmaceutical composition of claim 12, comprising tablets having a hardness about 5 to 20 kiloponds.

16. The pharmaceutical composition of claim 12, comprising primidone particles having a particle size D₉₀ no greater than about 15 μm.

17. The pharmaceutical composition of claim 16, wherein primidone particles have a Carr index at least about 20 percent.

18. The pharmaceutical composition of claim 16, comprising tablets having a hardness about 5 to 30 kiloponds.

19. The pharmaceutical composition of claim 16, comprising tablets having a hardness about 5 to 20 kiloponds.
